(19) United States
(12) Patent Application Publication
(10) Pub. No.: US 2023/0033606 A1
(43) Pub. Date: Feb. 2, 2023

(54) MULTI-STRAIN PROBIOTIC COMPOSITION AND ITS USE

(71) Applicant: METAGENICS, INC., Aliso Viejo, CA (US)

(72) Inventors: Noelle Marie PATNO, Aliso Viejo, CA (US); Jennifer Joan RYAN, Portland, OR (US)

(21) Appl. No.: 17/784,154

(22) PCT Filed: Dec. 17, 2020
(86) PCT No.: PCT/US2020/065590

§ 371 Date: Jun. 10, 2022

Related U.S. Application Data
(60) Provisional application No. 62/949,227, filed on Dec. 17, 2019.

(51) Int. Cl. A61K 35/744; A23L 33/135; A61P 1/00

(52) U.S. Cl. CPC A61K 35/744; A23L 33/135; A61P 1/00

(57) ABSTRACT

A probiotic composition is provided. The probiotic composition comprises different probiotic strains. The probiotic composition generally comprises: Bifidobacterium lactis; Lactobacillus acidophilus; Lactobacillusplantarum; Lactobacillus rhamno-sus; and Lactobacillusparacasei. The composition comprising suchprobiotic strains has abeneficial effect onthe intestinal microbiome diversity, improves the Firmicutes/Bacteroidetes ratio, and encourages growth of beneficial microbiota not contained in the composition itself. Also provided herein are uses of the probiotic composition.

### CROSS-REFERENCE TO RELATED APPLICATIONS

This application claims priority to and all advantages of U.S. Provisional Patent Application No. 62/949,227, filed on 17 Dec. 2019, the content of which is incorporated herein by reference.

### TECHNICAL FIELD

The present invention generally relates to a multi-strain probiotic composition having at least one synergistic effect in treating and/or preventing dysbiosis and maladies related thereto.

### BACKGROUND OF THE INVENTION

The intestinal microbiome has been associated with human health and/or disease in a number of large studies, including the Human Microbiome Project (Human Microbiome Project Consortium 2012) and the American Gut Project (McDonald et al. 2018). Two major measurements of the microbiome include diversity and dysbiosis. Less diversity has been associated with chronic diseases of metabolism and inflammation. Dysbiosis has become an appellation for the characterization of an unhealthy individual's intestinal bacteria, as an impaired derivative condition based on having fewer microbes or having more potentially pathogenic bacteria associated with healthy individuals' intestines or loss of the diversity that is relatively higher in the healthy state. In addition, the major shift in dietary patterns concomitant with the increased sterilization of the industrialized lifestyle has led to less exposure to bacteria at the same time as autoimmune and allergic conditions as well as stress, anxiety and maladaptive behaviors have been on the rise (Rook, Lowry, and Raison 2013).

Since the intestinal microbiome is under such investigation and moving toward being a proxy of health or disease, it has become an intermediate target for improving health not only of the gut but also the closely connected, vast number of nearby cells of the immune system as well as the connections to the central nervous system (i.e., the so-called gut-brain axis). The observation that certain types of bacterial populations, particularly the species from the phyla Firmicutes and Bacteroidetes that dominate the gut (Human Microbiome Project Consortium 2012), differ in abundance has been associated with health or disease. The ratio of these two phyla, Firmicutes/Bacteroidetes ratio, is considered an indicator of dysbiosis (Rowin et al. 2017).

Interventions that alter either the Firmicutes and Bacteroidetes have the potential to improve a Firmicutes/Bacteroidetes ratio. Similarly, the observation that diversity is relatively higher in healthier populations has generated the hypothesis that an intervention that increases the intestinal microbial diversity may improve health and reduce the risk for disease. These interventions sometimes call back to the ancestral days of more exposure to bacteria, soil, and different dietary components and lifestyle behaviors. Diet (Z. Xu and Knight 2015), fasting (Remely et al. 2015), and antibiotics (Dethlefsen et al. 2008), as well as other factors affect the diversity of the microbiome.

Thus, tools to manipulate the intestinal microbiome to improve dysbiosis or diversity, or rather, move the intestinal microbiome composition of bacteria from dysbiosis or low diversity to a profile more similar to a healthy individual's intestinal environment are desired to improve health.

While there have been assumptions and misperceptions that probiotics would be able to colonize and provide a source of probiotics to add to the diversity of the microbiome, there is a lack of sufficient evidence to support the idea. In fact, the definition of a probiotic which is internationally accepted, specifies that probiotics are living microorganisms that, in adequate amounts, provide clinically-effective results (Hill et al. 2014). Typically, the endpoints of probiotics have been for their clinical benefit (such as reduction in severity and duration of diarrhea or common cold symptoms) consistent with this definition as well. While the globally-understood definition of probiotic is based on clinical evidence, popular ideas still persist that the probiotic will alter the microbiome and/or potentially change the diversity, which seems to be the basis behind some practitioners' use of rotation of probiotic supplementation as well as using different types of bacteria or multiple and high quantities of bacteria. Despite this belief, there has been one probiotic strain demonstrated to affect microbiome diversity in an arteriosclerotic male population based on diversity calculated from an old method of analyzing bacterial DNA from the gut referenced in a patent (U.S. Pat. No. 8,586,026 B2). This patent covers that strain and a few strains of Lactobacillus plantarum for increasing the diversity of the gastrointestinal tract with the measurement of low bacterial diversity using the T-RFLP method for characterization of the DNA prior to calculating the Shannon and Weaver diversity index and the Simpson diversity index. The T-RFLP method, which only fragments the DNA and does not actually identify bacteria, is older technology which is not used as frequently since next generation sequencing has increased the speed and automation of full sequencing and thus more accurate methods, which provide different results from older and cruder methodology, are available to evaluate tools that affect the intestinal microbiome.

On the other hand, larger levers such as dietary ingredients including fiber and prebiotics have also been assumed to have a bigger impact on the intestinal microbiome, especially with support from the American Gut project's finding that a much higher vegetable intake is associated with greater diversity in samples from those individuals. Interventional studies provide a paucity of direct examples. One prebiotic actually showed that certain diversity indices, calculated and used after 16S rRNA DNA sequencing for bacterial identification, decreased following supplementation (Alfa et al. 2018). The ability to predictably alter the diversity of the microbiome and thereby improve the health of an individual remains elusive.

While one may expect a high dose multiple probiotic strain combination might increase beneficial bacteria populations, improve microbial diversity and reduce dysbiosis, scientific evidence remains lacking or absent. In addition, probiotic clinical studies have often been conducted without any measurement of the intestinal microbiome or its diversity, and showing only modest changes to microbiota or none at all.

As a result, it has become apparent that combinations of probiotic strains and higher doses of probiotics may not necessarily be “better” or more effective than single strain at achieving the desired effect of reducing abdominal pain or other clinical symptom relief targets. Clinically-effective amounts are derived by experimentation and they may occur based on a synergistic combination of certain amounts of multiple strains of bacteria. Limited published research compares strains to each other and often these studies have different study designs, making direct comparisons of results nearly impossible. As an example of strain importance, Lactobacillus plantarum 299v (Ducrotté, Sawant, and Jayanthi 2012) was beneficial while another strain in the same species (MF1298) was apparently detrimental for irritable bowel syndrome (IBS) (Ligaarden et al. 2010). There is a lack of research on microbiome diversity shifts associated with amelioration of symptoms.

Therefore, a multi-strain, high quantity probiotic product with exhibiting microbiome shifts or enhancing diversity would be novel and welcome, since many marketed combination products have no such evidence behind them. From the perspective of meeting the clinical effectiveness requirement of the probiotic definition, starting with a combination of probiotics that have clinical efficacy provides a basis for an effective product. The notion behind the combination is that these probiotic strains have already shown efficacy and would still be clinically efficacious in a combination. An effective product of multiple probiotic strains would guarantee CFU in clinically demonstrated effective amounts for each strain and/or as a combination.

Another reason for increased diversity or ameliorating dysbiosis is to address specific types of bacterial populations associated with healthy versus diseased states. A number of bacterial species are not available as probiotics, and the absence of a number of these species in the intestinal microbiota is indicative of disease. For example, Faecalibacterium prausnitzii and Akkermansia muciniphila are absent or at low levels in diseased individuals and thus implicated to be associated with health. Specifically, low levels of Faecalibacterium prausnitzii are associated with a number of diseases, such as inflammatory bowel disease (IBD) (Cao, Shen, and Ran 2014), irritable bowel syndrome (IBS) (Lyra et al. 2009), Celiac disease (De Palma, Collins, and Bercik 2014), and others while low levels of Akkermansia muciniphila are associated with inflammatory bowel disease (Lopez-Siles et al. 2012), obesity (Dao et al. 2016), metabolic syndrome (Dao et al. 2016), and others. While there may be a desire to administer F. prausnitzii and Akkermansia muciniphila as probiotic supplements, they are unavailable as probiotics, due to their sensitivity to oxygen and associated challenges to culture, manufacture or store them.

In the treatment of dysbiosis and its related maladies, it is desired that a composition be developed that can shift the gut microbiota (away from both those microorganisms indicative of disease and unhealthy imbalances of microorganisms which are known to characterize disease), enhance diversity, improve Firmicutes/Bacteroidetes ratio (an indicator of dysbiosis), and increase key species that are associated with health in order to improve gastrointestinal, immune and stress response symptoms to reduce and to prevent disease.

### PRIOR ART DOCUMENTS

### Patent Documents

Patent Document 1: U.S. Pat. No. 8,586,026 B2

Non-patent Literature (NPL) Documents

NPL Document 1:
Alfa, Michelle J., David Strang, Paramjit S. Tappia, Nancy Olson, Pat DeGagne, David Bray, Brenda-Lee Murray, and Brett Hiebert. 2018. “A Randomized Placebo Controlled Clinical Trial to Determine the Impact of Digestion Resistant Starch MSPrebiotic® on Glucose, Insulin, and Insulin Resistance in Elderly and Mid-Age Adults.” Frontiers in Medicine 4. https://doi.org/10.3389/fmed.2017.00260.

NPL Document 2:
Canani, Roberto Berni, Rita Nocerino, Gianluca Terrin, Anna Coruzzo, Linda Cosenza, Ludovica Leone, and Riccardo Troncone. 2012. “Effect of Lactobacillus GG on Tolerance Acquisition in Infants with Cow's Milk Allergy: A Randomized Trial.” Journal ofAllergy and Clinical Immunology 129 (2): 580-582.e5. https://doi.org/10.1016/j.jaci.2011.10.004.

NPL Document 3:
Cao, Yuan, Jun Shen, and Zhi Hua Ran. 2014. “Association between Faecalibacterium Prausnitzii Reduction and Inflammatory Bowel Disease: A Meta-Analysis and Systematic Review of the Literature.” Research article. Gastroenterology Research and Practice. 2014. https://doi.org/10.1155/2014/872725.

NPL Document 4:
Carding, Simon, Kristin Verbeke, Daniel T. Vipond, Bernard M. Corfe, and Lauren J. Owen. 2015. “Dysbiosis of the Gut Microbiota in Disease.” Microbial Ecology in Health and Disease 26 (February). https://doi.org/10.3402/mehd.v26.26191.

NPL Document 5:
Chen, Lihong, Courtney Reynolds, Robert David, and Amy Peace Brewer. 2019. “Development of an Index Score for Intestinal Inflammation-Associated Dysbiosis Using Real-World Stool Test Results.” Digestive Diseases and Sciences, September. https://doi.org/10.1007/si0620-019-05828-8.

NPL Document 6:
Dao, Maria Carlota, Amandine Everard, Judith Aron-Wisnewsky, Nataliya Sokolovska, Edi Prifti, Eric O Verger, Brandon D Kayser, et al. 2016. “Akkermansia Muciniphila and Improved Metabolic Health during a Dietary Intervention in Obesity: Relationship with Gut Microbiome Richness and Ecology.” Gut 65 (3): 426-36. https://doi.org/10.1136/gutjnl-2014-308778.

NPL Document 7:
De Palma, Giada, Stephen M Collins, and Premysl Bercik. 2014. “The Microbiota-Gut-Brain Axis in Functional Gastrointestinal Disorders.” Gut Microbes 5 (3): 419-29. https://doi.org/10.4161/gmic.29417.

NPL Document 8:
Dethlefsen, Les, Sue Huse, Mitchell L Sogin, and David A Relman. 2008. “The Pervasive Effects of an Antibiotic on the Human Gut Microbiota, as Revealed by Deep 16S RRNA Sequencing.” PLoS Biology 6 (11). https://doi.org/10.1371/journal.pbio.0060280.

NPL Document 9:
Doron, Shira, and David R. Snydman. 2015. “Risk and Safety of Probiotics.” Clinical Infectious Diseases: An Official Publication ofthe Infectious Diseases Society ofAmerica 60 (Suppl 2): S129-34. https://doi.org/10.1093/cid/civ085.

NPL Document 10:
Ducrotté, Philippe, Prabha Sawant, and Venkataraman Jayanthi. 2012. “Clinical Trial: Lactobacillus Plantarum 299v (DSM 9843) Improves Symptoms of Irritable Bowel Syndrome.” World Journal of Gastroenterology: WJG 18 (30): 4012-18. https://doi.org/10.3748/wjg.vl8.i30.4012.

NPL Document 11:
Dudzicz, Sylwia, Agata Kujawa-Szewieczek, Katarzyna Kwiecien, Andrzej Wiecek, and Marcin Adamczak. 2018. “Lactobacillus Plantarum 299v Reduces the Incidence of Clostridium Difficile Infection in Nephrology and Transplantation Ward-Results of One Year Extended Study.” Nutrients 10 (11): 1574. https://doi.org/10.3390/nul0111574.

NPL Document 12:
Finotello, Francesca, Eleonora Mastrorilli, and Barbara Di Camillo. 2016. “Measuring the Diversity of the Human Microbiota with Targeted Next-Generation Sequencing.” Briefings in Bioinformatics, December, bbwl19. https://doi.org/10.1093/bib/bbwl19.

NPL Document 13:
Forssten, S. D., and A. C. Ouwehand. 2017. “Simulating Colonic Survival of Probiotics in Single-Strain Products Compared to Multi-Strain Products.” Microbial Ecology in Health and Disease 28 (1): 1378061. https://doi.org/10.1080/16512235.2017.1378061.

NPL Document 14:
Gill, H. S., K. J. Rutherfurd, J. Prasad, and P. K. Gopal. 2000. “Enhancement of Natural and Acquired Immunity by Lactobacillus Rhamnosus (HN001), Lactobacillus Acidophilus (HN017) and Bifidobacterium Lactis (HN019).” British Journal ofNutrition 83 (02): 167. https://doi.org/10.1017/S0007114500000210.

NPL Document 15:
Gill, Harsharnjit S., Kay J. Rutherfurd, Martin L. Cross, and Pramod K. Gopal. 2001. “Enhancement of Immunity in the Elderly by Dietary Supplementation with the Probiotic Bifidobacterium Lactis HN019.” The American Journal of Clinical Nutrition 74 (6): 833-39. https://doi.org/10.1093/ajcn/74.6.833.

NPL Document 16:
Hill, Colin, Francisco Guarner, Gregor Reid, Glenn R. Gibson, Daniel J. Merenstein, Bruno Pot, Lorenzo Morelli, et al. 2014. “Expert Consensus Document: The International Scientific Association for Probiotics and Prebiotics Consensus Statement on the Scope and Appropriate Use of the Term Probiotic.” Nature Reviews Gastroenterology & Hepatology 11 (8): 506-14. https://doi.org/10.1038/nrgastro.2014.66.

NPL Document 17:
Human Microbiome Project Consortium. 2012. “Structure, Function and Diversity of the Healthy Human Microbiome.” Nature 486 (7402): 207-14. https://doi.org/10.1038/nature11234.

NPL Document 18:
Kaur, Sumanpreet, Preeti Sharma, Namarta Kalia, Jatinder Singh, and Sukhraj Kaur. 2018. “Anti-Biofilm Properties of the Fecal Probiotic Lactobacilli Against Vibrio Spp.” Frontiers in Cellular and Infection Microbiology 8 (April). https://doi.org/10.3389/fcimb.2018.00120.

NPL Document 19:
Khan, Israr, Naeem Ullah, Lajia Zha, Yanrui Bai, Ashiq Khan, Tang Zhao, Tuanjie Che, and Chunjiang Zhang. 2019. “Alteration of Gut Microbiota in Inflammatory Bowel Disease (IBD): Cause or Consequence? IBD Treatment Targeting the Gut Microbiome.” Pathogens 8 (3). https://doi.org/10.3390/pathogens8030126.

NPL Document 20:
Korada, Siva K., Nagendra S. Yarla, Vijayendra Mishra, Mohamed A. Daim, Bechan Sharma, Ashraf Gm, Reggi R., Palmery M, Ilaria Peluso, and Mohammad A. Kamal. 2019. “Single Probiotic versus Multiple Probiotics -A Debate On Current Scenario for Alleviating Health Benefits.” Current Pharmaceutical Design 24 (35): 4150-53. https://doi.org/10.2174/1381612824666181012124136.

NPL Document 21:
Kravitz, Richard L., and Naihua Duan. 2014. “Design and Implementation of N-of-1 Trials: A User's Guide.” Agency for Healthcare Research and Quality 13 (14): 94.

NPL Document 22:
Larsen, Nadja, Finn K. Vogensen, Rikke Gobel, Kim F. Michaelsen, Waleed Abu Al-Soud, Soren J. Sorensen, Lars H. Hansen, and Mogens Jakobsen. 2011. “Predominant Genera of Fecal Microbiota in Children with Atopic Dermatitis Are Not Altered by Intake of Probiotic Bacteria Lactobacillus Acidophilus NCFM and Bifidobacterium Animalis Subsp. Lactis Bi-07: Probiotics and Intestinal Microbiota.” FEMS Microbiology Ecology 75 (3): 482-96. https://doi.org/10.1111/j.1574-6941.2010.01024.x.

NPL Document 23:
Li, Xuanji, Asker Daniel Brejnrod, Madeleine Ernst, Martin RykTr, Jakob Herschend, Nanna Mee Coops Olsen, Pieter C. Dorrestein, Christopher Rensing, and Soren Johannes Sorensen. 2019. “Heavy Metal Exposure Causes Changes in the Metabolic Health-Associated Gut Microbiome and Metabolites.” Environment International 126 (May): 454-67. https://doi.org/10.1016/j.envint.2019.02.048.

NPL Document 24:
Ligaarden, Solveig C., Lars Axelsson, Kristine Naterstad, Stian Lydersen, and Per G. Farup. 2010. “A Candidate Probiotic with Unfavourable Effects in Subjects with Irritable Bowel Syndrome: A Randomised Controlled Trial.” BMC Gastroenterology 10 (1): 16. https://doi.org/10.1186/1471-230X-10-16.

NPL Document 25:
Liu, Shan, PengWei Hu, Xiaoxin Du, Tao Zhou, and Xiaofang Pei. 2013. “Lactobacillus Rhamnosus GG Supplementation for Preventing Respiratory Infections in Children: A Meta-Analysis of Randomized, Placebo-Controlled Trials.” Indian Pediatrics 50 (4): 377-81. https://doi.org/10.1007/s13312-013-0123-z.

NPL Document 26:
Liu, Yue, Glenn R. Gibson, and Gemma E. Walton. 2016. “An In Vitro Approach to Study Effects of Prebiotics and Probiotics on the Faecal Microbiota and Selected Immune Parameters Relevant to the Elderly.” PLoS ONE 11 (9). https://doi.org/10.1371/journal.pone.0162604.

NPL Document 27:
Lopez-Siles, Mireia, Tanweer M. Khan, Sylvia H. Duncan, Hermie J. M. Harmsen, L. Jesns Garcia-Gil, and Harry J. Flint. 2012. “Cultured Representatives of Two Major Phylogroups of Human Colonic Faecalibacterium Prausnitzii Can Utilize Pectin, Uronic Acids, and Host-Derived Substrates for Growth.” Applied and Environmental Microbiology 78 (2): 420-28. https://doi.org/10.1128/AEM.06858-11.

NPL Document 28:
Lyra, Anna, Teemu Rinttili, Janne Nikkili, Lotta Krogius-Kurikka, Kajsa Kajander, Erja Malinen, Jaana Mitt6, Laura Mikeli, and Airi Palva. 2009. “Diarrhoea-Predominant Irritable Bowel Syndrome Distinguishable by 16S RRNA Gene Phylotype Quantification.” World Journal of Gastroenterology: WJG 15 (47): 5936-45. https://doi.org/10.3748/wjg.15.5936.

NPL Document 29:
Maier, Tanja V., Marianna Lucio, Lang Ho Lee, Nathan C. VerBerkmoes, Colin J. Brislawn, Jorg Bernhardt, Regina Lamendella, et al. 2017. “Impact of Dietary Resistant Starch on the Human Gut Microbiome, Metaproteome, and Metabolome.” MBio 8 (5): e01343-17. https://doi.org/10.1128/mBio.01343-17.

NPL Document 30:
McDonald, Daniel, Embriette Hyde, Justine W. Debelius, James T. Morton, Antonio Gonzalez, Gail Ackermann, Alexander A. Aksenov, et al. 2018. “American Gut: An Open Platform for Citizen Science Microbiome Research.” MSystems 3 (3). https://doi.org/10.1128/mSystems.00031-18.

NPL Document 31:
Musa, Md. Abu, Mamun Kabir, Md. Iqbal Hossain, Emtiaz Ahmed, Abdullah Siddique, Humaira Rashid, Mustafa Mahfuz, et al. 2019. “Measurement of Intestinal Permeability Using Lactulose and Mannitol with Conventional Five Hours and Shortened Two Hours Urine Collection by Two Different Methods: HPAE-PAD and LC-MSMS.” PLoS ONE 14 (8). https://doi.org/10.1371/journal.pone.0220397.

NPL Document 32:
Paineau, Damien, Didier Carcano, Greg Leyer, Sylviane Darquy, Marie-Alexandra Alyanakian, Guy Simoneau, Jean-FranA§ ois Bergmann, Dominique Brassart, Francis Bornet, and Arthur C. Ouwehand. 2008. “Effects of Seven Potential Probiotic Strains on Specific Immune Responses in Healthy Adults: A Double-Blind, Randomized, Controlled Trial.” FEMS Immunology & Medical Microbiology 53 (1): 107-13. https://doi.org/10.1111/j.1574-695X.2008.00413.x.

NPL Document 33:
Queiroz, Camila AA de, Said Gongalves C Fonseca, Priscila B Frota, Italo L Figueiredo, Karoline S Aragio, Carlos Emanuel C Magalhies, Cibele BM de Carvalho, et al. 2014. “Zinc Treatment Ameliorates Diarrhea and Intestinal Inflammation in Undernourished Rats.” BMC Gastroenterology 14 (August): 136. https://doi.org/10.1186/1471-230X-14-136.

NPL Document 34:
Remely, Marlene, Berit Hippe, Isabella Geretschlaeger, Sonja Stegmayer, Ingrid Hoefinger, and Alexander Haslberger. 2015. “Increased Gut Microbiota Diversity and Abundance of Faecalibacterium Prausnitzii and Akkermansia after Fasting: A Pilot Study.” Wiener Klinische Wochenschrift 127 (9-10): 394-98. https://doi.org/10.1007/s00508-015-0755-1.

NPL Document 35:
Rook, Graham A. W., Christopher A. Lowry, and Charles L. Raison. 2013. “Microbial ‘Old Friends’, Immunoregulation and Stress Resilience.” Evolution, Medicine, and Public Health 2013 (1): 46-64. https://doi.org/10.1093/emph/eot004.

NPL Document 36:
Rowin, Julie, Yinglin Xia, Barbara Jung, and Jun Sun. 2017. “Gut Inflammation and Dysbiosis in Human Motor Neuron Disease.” Physiological Reports 5 (18). https://doi.org/10.14814/phy2.13443.

NPL Document 37:
Scher, Jose U., Carles Ubeda, Alejandro Artacho, Mukundan Attur, Sandrine Isaac, Soumya M.

Reddy, Shoshana Marmon, et al. 2015. “Decreased Bacterial Diversity Characterizes an Altered Gut Microbiota in Psoriatic Arthritis and Resembles Dysbiosis of Inflammatory Bowel Disease.” Arthritis & Rheumatology (Hoboken, N.J.) 67 (1): 128-39. https://doi.org/10.1002/art.38892.

NPL Document 38:
Sokol, Harry, B6n6dicte Pigneur, Laurie Watterlot, Omar Lakhdari, Luis G. Bermndez-Humarin, Jean-Jacques Gratadoux, S6bastien Blugeon, et al. 2008. “Faecalibacterium Prausnitzii Is an Anti-Inflammatory Commensal Bacterium Identified by Gut Microbiota Analysis of Crohn Disease Patients.” Proceedings of the National Academy of Sciences ofthe United States ofAmerica 105 (43): 16731-36. https://doi.org/10.1073/pnas.0804812105.

NPL Document 39:
Xu, Ling-Zhi, Li-Tao Yang, Shu-Qi Qiu, Gui Yang, Xiang-Qian Luo, Bei-Ping Miao, Xiao-Rui Geng, et al. 2016. “Combination of Specific Allergen and Probiotics Induces Specific Regulatory B Cells and Enhances Specific Immunotherapy Effect on Allergic Rhinitis.” Oncotarget 7 (34): 54360-69. https://doi.org/10.18632/oncotarget.10946.

NPL Document 40:
Xu, Zhenjiang, and Rob Knight. 2015. “Dietary Effects on Human Gut Microbiome Diversity.” The British Journal of Nutrition 113 (Suppl 0): S1-5. https://doi.org/10.1017/S0007114514004127.

NPL Document 41:
Zhang, Haoze, Ping Li, Di Wu, Dong Xu, Yong Hou, Qian Wang, Mengtao Li, et al. 2015. “Serum IgG Subclasses in Autoimmune Diseases.” Medicine 94 (2). https://doi.org/10.1097/MD.0000000000000387.

### SUMMARY OF THE INVENTION

Provided herein is a probiotic composition. The probiotic composition generally comprises: Bifidobacterium lactis; Lactobacillus acidophilus; Lactobacillus plantarum; Lactobacillus rhamnosus; and Lactobacillus paracasei. Also provided herein are methods and uses associated with the probiotic composition. For example, the probiotic composition can be used to treat and/or prevent dysbiosis and/or maladies related thereto.

The inventors discovered a particular combination of probiotic strains in the composition of this disclosure that has a surprising beneficial effect on the intestinal microbiome. Without being bound or limited to any particular theory, it is believed that this combination enhances diversity, improves the Firmicutes/Bacteroidetes ratio (an indicator of dysbiosis), and unexpectedly encourages growth of microbiota not contained in the composition of this disclosure itself, and demonstrates improvement in gastrointestinal health from a clinical perspective, as well as other beneficial effects, as described herein.

An objective of the present disclosure is to provide a means of improving gastrointestinal and immune and metabolic health through stress resilience such as addressing low bacterial diversity or dysbiosis conditions associated with inflammatory and metabolic dysregulation. An additional objective of the present disclosure includes improvement of the intestinal microbiome's diversity indices, including an increase in populations of specific bacteria associated with health.

A further objective of the present disclosure is to produce beneficial metabolites and interact with immune cells in the intestine. A yet further objective of the present disclosure is to provide a treatment for any of the following; irregularity, poor frequency of stools, resolving loose stools to more formed stools, irritable bowel syndrome, constipation, diarrhea, flatulence, bloating, modulating inflammation of the intestine associated with diseases such as inflammatory bowel disease including Crohn's or Ulcerative colitis and the manifestation of pouchitis, celiac disease and other intestinal disorders, malabsorption and digestive disorders, abdominal pain, cramping, abdominal motility issues, compromised immune function including increased susceptibility or occurrence of upper respiratory tract infections/the common cold, adjuvant support for immunizations or any immunotherapy, allergen challenges, environmental toxins, individuals particularly susceptible to stress-induced headaches and abdominal pain, and/or relief from anxiety or depression comorbid with gastrointestinal (GI) distress.

### DETAILED DESCRIPTION OF THE INVENTION

### Definitions

Several terms used herein are defined here for the reader, and for the understanding of the claims related to this disclosure:

“Bacterial diversity” is a measurement of the abundance, relative or absolute, of different bacteria (whether strain, species, genus or family identified) which is called “richness”, and/or the distribution of that abundance (“evenness”), which may be measured by methods known in the art, such as the Shannon diversity index, the Chaol, the Simpson index, the inverse Simpson index, or other measurements known in the art that take into account types and/or amounts of different bacteria in a microbiotic sample. As it relates to this disclosure, such a sample would be taken from the intestine, e.g., from a feces sample. In other, related cases, such a sample could be taken via an oral sample or endoscopic samples from the mouth through to the anus.

“Identification methods of intestinal gut bacteria”, as referred to herein, may include sequencing the bacteria by PCR or 16S rRNA or shotgun sequencing or flow cytometry or other methods known in the art. Harmonization of standards and methodologies for identification of bacteria in the intestine is ongoing, and multiple methodologies are currently available. As a result, some variations in results between methods is expected. For example, certain histological methods previously used may be outmoded, but may still be used.

“Low bacterial diversity” is a term of art used to define a low relevant index of diverse microbes present in the intestine measured as per the literature and state-of-the-art technology and scientific evidence. For example, C. difficile is present in many individuals, but when this microbe overwhelms the rest of the diverse microbiota in the GI tract, this change (referred to as infection) can be life threatening.

“Beneficial metabolites”, as used herein, refers to any compound produced by bacteria in the intestine (from mouth to anus) such as short chain fatty acids (including but not limited to acetate, propionate, butyrate) or secondary bile acids or other compounds.

“Dysbiosis-related conditions” refers to conditions associated with an imbalance in the microbiota, which may be characterized by a loss of beneficial bacteria, an overgrowth of potentially pathogenic bacteria, or a loss of overall microbial diversity (Carding et al. 2015). For example, inflammatory bowel diseases such as Crohn's (Lopez-Siles et al. 2012; Sokol et al. 2008) and Ulcerative colitis (Khan et al. 2019), Parkinson's disease, immune-mediated such as psoriasis (Scher et al. 2015), and even metabolic disease and cognitive dysfunction (Carding et al. 2015) are all related to dysbiosis.

“Inflammatory and metabolic dysregulation” refers to altered signaling and/or biomarker changes in the inflammatory and metabolic cascades and/or biochemical signaling. Such changes include, but are not limited to upregulation of cytokines associated with inflammation, changes to glucose levels and/or insulin responses that are altered from normal, healthy physiological activity.

“Intestinal microbiome's diversity indices” refers, as described above, to such measures of diversity, such as (but not limited to) known indices, like the Shannon diversity index, the Chaol, the Simpson index, the inverse Simpson index, or other measurements that take into account types and/or amounts of different bacteria, e.g., any of the diversity indices described in (Finotello, Mastrorilli, and Di Camillo 2016).

“Prebiotic” is a substrate that is consumed by or promotes the growth of bacteria in the intestine or gastrointestinal tract of the individual consuming the prebiotic. This growth of bacteria provides a health benefit to the individual or improves measures or metrics associated with gut health. Examples of such measurements or metrics include measurement of inulin, FOS (fructooligosaccharides), GOS (galactooligosaccharides), and HMOs (human milk oligosaccharides) and other substrates.

“Probiotic” refers to live microbes that, when consumed at adequate amounts, provide a health benefit to the host. Often these microbes are similar to microbes resident in the human gut, but may be found in very small numbers (e.g., as evidenced by dysbiosis), or they may require to process prebiotics or other substrates to encourage growth of other microbes.

“Stress resilience” refers to the ability to respond and recover from stress (e.g., return from inflammatory and/or reactive state to return to normal state (e.g., homeostasis). In this context, for example, the stress of a diet, colonoscopy or other intestinal procedure, the bowel preparation for the colonoscopy or other laxatives, stresses of life that might induce diarrhea or other GI disturbances would then be returned to a normal state (e.g., more regular bowel movements, normal or a condition prior to the stress. Resilience would be measured by the reduction in time to return to a condition within an insignificant difference from or within a patient's acceptable difference from their base case and/or condition prior to stress.

“Biofilm” refers to a layer or “film” of biologically-active organisms formed by adhesion of the bacteria or other microbes to the intestinal cells and/or intestinal wall. Such biofilm can act as a protective layer, as a symbiotic component or as a biologically active part of the digestive system.

“Intestinal permeability” refers to a rate or flux or relative amount transferred of molecules, microbes, compounds, substrates from the intestinal lumen across the intestinal barrier into the lymph/blood/peritoneal space as measured by a number of methods, such as by lactulose/mannitol measurements, serum measurements of LPS, zonulin or other components from intestinal lumen, or in the lymphatic fluid. Because the intestine is selective by nature, and a healthy gut only allows certain components to pass freely into the body, “intestinal permeability” is often used to describe dysbiosis or illness. As a result, intestinal permeability in its worst form is often referred to by hyper-permeability or “leaky gut”, and indicates the inability of the intestine to be selective, and, in effect allows toxins into the body that would otherwise be excluded and excreted.

“CFU”, as used herein, means a “colony forming unit”, which is generally used to describe numbers of viable microbes, bacterium or organisms (“probiotic”) in a given formulation.

Manufacturers may formulate to a higher CFU number, but label the product with a lower number that reflects the amount that an individual likely will receive when ingesting the probiotic formulation, knowing that the viable CFU decrease over time. It is not uncommon for formulations to have up to four times the labeled CFU potency at manufacture due to room temperature storage, combinations with other probiotics, exposure to air, etc. In addition, combinations may be adjusted to fit into a specific capsule and adjusted for cost as well. Though higher CFU numbers may be used in packaging, safety at that higher CFU amount is evaluated to assure no health or safety issue arises.

As used herein, the terms “treatment” and “treat” refer to and encompass prophylactic (i.e., preventive), modifying, and curative treatments. As such, these terms include treatment of subjects/patients (e.g. humans) at risk of contracting a disease or suspected to have contracted a disease, as well as subjects/patients who are ill or have been diagnosed as suffering from a disease or medical condition.

As used herein, the term “therapeutically effective amount” relates to an amount (i.e., a quantity) of a composition required to achieve a particular therapeutic and/or prophylactic effect, such as in treating a patient. Likewise, as used herein, the term “physiologically effective amount” relates to an amount of a composition required to achieve a desired physiological effect. As will be understood by one of skill in the art, such effective amounts are typically measured and/or expressed in terms of g/day, or a derivative thereof (e.g. mg/day).

As used herein, the term “foodstuff” refers to a material that may be used as a food. As such, in certain instances the term foodstuff is used to describe a composition that may be consumed (e.g. by eating) by a living organism (e.g. a mammal), such as for nourishment and/or sustenance.

As used herein, the term “beverage” refers to a potable liquid or other non-solid composition. As such, in certain instances the term beverage is used to describe a non-solid (e.g. liquid, slurry, suspension, etc.) composition that may be consumed by a living organism for nourishment and/or sustenance. As such, in particular instances the terms “beverage” and “foodstuff” may overlap. In certain instances, the term “nutritional composition” is used to describe a foodstuff and/or beverage formulation that can be eaten or drunk by a human subject for nutrition.

As used herein, the term “functional food additive” refers to an ingredient, additive, component, or supplement suitable for incorporation in a foodstuff and/or beverage to confer a technical, nutritional, and/or health benefit (i.e., a function) to a host that consumes the foodstuff and/or beverage. The “functional food additive” can be added to different types of food including, but not limited to, medical foods, dietetic foods, and supplements.

As used herein, the term “medical food” is typically used to refer to a food for a special dietary use, such as a food formulated for dietary management of a medical condition (e.g. based upon scientific or medical evaluation). However, it is to be appreciated that the term “medical food” may have one or more particular definitions depending on, for example, geographic location, specific use, regulatory agency, and the like. For example, in certain cases, the term medical food may be defined as a food which is formulated to be consumed or administered enterally under the supervision of a physician and which is intended for the specific dietary management of a disease or condition for which distinctive nutritional requirements, based on recognized scientific principles, are established by medical evaluation (see, e.g., section 5(b) of the Orphan Drug Act (21 U.S.C. 360ee (b) (3)), which is incorporated herein by reference). In these or other instances, the term medical food may be defined as a food for special dietary use as a food that has been specially processed or formulated to meet the particular requirements of a person: (a) in whom a physical or physiological condition exists as a result of a disease, disorder, or injury; or (b) for whom a particular effect, including but not limited to weight loss, is to be obtained by a controlled intake of food (see, e.g., section B.24.001 of the Canadian Food and Drug Regulations (FDR, C.R.C., c. 870)(as amended 13 Jun. 2017)), which is incorporated herein by reference).

As used herein, the term “supplement” relates to a nutritional supplement which is a concentrated source of nutrient or alternatively other substances with a nutritional or physiological effect whose purpose is to supplement the normal diet.

Probiotic Composition

The probiotic composition comprises multiple probiotic bacterial strains. Thus, the probiotic composition can be referred to as a multi-strain probiotic composition. The probiotic composition may simply be referred to herein as the composition.

In various embodiments, the probiotic composition comprises: Bifidobacterium lactis; Lactobacillus acidophilus; Lactobacillus plantarum; Lactobacillus rhamnosus; and Lactobacillus paracasei. In further embodiments, the probiotic composition comprises at least two different strains of Lactobacillus rhamnosus. In further or alternate embodiments, the probiotic composition comprises at least two different strains of Bifidobacterium lactis. In yet further or alternate embodiments, the probiotic composition comprises at least three different strains of Bifidobacterium lactis.

In certain embodiments, the probiotic composition comprises: at least two or two different strains of Bifidobacterium lactis; at least one or one strain of Lactobacillus acidophilus; at least one or one strain of Lactobacillus plantarum; at least two or two different strains of Lactobacillus rhamnosus; and at least one or one strain of Lactobacillus paracasei. In further embodiments, the probiotic composition comprises: at least three or three different strains of Bifidobacterium lactis; at least one or one strain of Lactobacillus acidophilus; at least one or one strain of Lactobacillus plantarum; at least two or two different strains of Lactobacillus rhamnosus; and at least one or one strain of Lactobacillus paracasei. Specific suitable strains for each probiotic are described further below. In specific embodiments, the composition is free of or substantially free of other probiotic bacterial strains. Said another way, in certain embodiments, the composition consists essentially of, or consists of, the probiotics (e.g. as noted in Table 1 and Table 2 below) as actives of the composition.

Each of the probiotic bacterial strains can be used in various amounts. That said, certain minimum amounts are useful as described herein, e.g. in the EXAMPLES section.

In various embodiments, the composition includes one or more additives that are generally used for conventional compositions, including those adapted for oral administration. In certain embodiments, the composition of this disclosure comprises one or more inactive ingredients. If utilized, the inactive ingredients are different from the probiotic bacterial strains.

Inactive ingredients are understood in the art and are different from active ingredients.

Examples of inactive ingredients include, but are not limited to, flavorings; carob; corn syrups, such as hydrolyzed corn syrup solids; cellulose, such as methyl cellulose, hydroxypropyl methyl cellulose, carboxy methyl cellulose, microcrystalline cellulose, and powdered cellulose; fructose; maltodextrin and maltol, such as natural maltol; sorbitol; preservatives; alcohols, such as ethanol, propyl alcohol and benzyl alcohol; glycerin; potassium sorbate; sodium benzoate; binders; flow agents; stearates, such as calcium stearate, magnesium stearate, and sodium magnesium stearate; dicalcium phosphate; glyceryl triacetate; vegetable oils, such as hydrogenated vegetable oils; mineral oils; water; silicones, such as silicone oils; silicon dioxide; stearic acid; waxes, such as carnauba wax and beeswax; starches, such as corn starch and potato starch; fatty esters and fatty alcohols; glycols and polyglycols; and combinations thereof.

If utilized to form the composition, the inactive ingredient(s) can be used in various amounts. This disclosure is not limited to a particular inactive ingredient or amount thereof.

The composition can be prepared using various methods understood in the art. For example, probiotic bacterial strains of the composition, and optionally one or more inactives, can be mixed or blended and compressed or compounded utilizing various techniques understood in the art. The composition of this disclosure is not limited to a particular order of manufacturing steps or method of manufacture.

The composition can be in various forms. Examples of suitable forms include solids, gels and liquids. Typically, the composition is solid. For example, the composition can be in the form of a pill, including tablets, capsules, and caplets. In general, each of these terms can be used interchangeable in the art, e.g. tablet for pill or vice versa. Other than the probiotic bacterial strains (i.e., the “actives” or “active ingredients”), the composition can include inactives (or “inactive ingredients”) including, but not limited to, excipients, such as diluents and binders; granulating agents; glidants (or flow aids); fillers; lubricants; preservatives; stabilizers; coatings; disintegrants; sweeteners or flavors; and pigments. Further examples of inactive ingredients are described above. In general, a number and quantity of excipients should be kept at a minimum as long as active ingredients are properly delivered. This is because subjects/consumers tend to prefer smaller tablets for easier consumption.

The composition can be in powder form, or pressed or compacted from a powder into a solid dose. A coating, e.g. polymer coating, may be used to make the tablet smoother and easier to swallow, to control release rate of the probiotic bacterial strains, to increase resiliency (or shelf life), and/or to enhance appearance. Other suitable oral forms of the composition include syrups, elixirs, suspensions, emulsions, and powders (e.g. for use in making foods, drinks, etc.). Further non-limiting embodiments of the composition of this disclosure are described hereafter.

In some embodiments, the composition is adapted to be consumed as a liquid. For example, the composition may be a dry powder that is combined with a consumable liquid (e.g. water) to form a consumable liquid solution, suspension, or emulsion comprising the composition.

Likewise, the composition may be adapted to be mixed with a foodstuff or beverage. As such, in some embodiments, the composition is, alternatively is a component of, a foodstuff or beverage.

In these or other embodiments, the composition may be further defined as a food additive.

Accordingly, it is to be appreciated that certain aspects of the present embodiments include the use of the composition as a food additive, and the use of the composition in methods of preparing foodstuff and/or beverages.

In some embodiments, the foodstuff or beverage comprising the composition is further defined as a nutritional composition. In these or other embodiments, the nutritional composition is in the form of a dry food concentrate, which may be mixed with liquid or food and subsequently consumed. It is to be appreciated that the nutritional composition is distinguished from a vaccine, and the compositions described herein may be free, alternatively substantially free, from a vaccine.

In some embodiments, the foodstuff or beverage is further defined as a medical food. As such, it is to be appreciated that the medical food comprises the composition, and may be the same as or different from the nutritional composition described above.

Typically, the composition is administered orally. As such, the composition may be referred to as an oral composition. However, rectal and/or enteral administration may also be used.

The composition can be administered in various amounts. The composition may be administered as needed, daily, several times per day or in any suitable regimen such that the desired outcome is achieved. In the method of this disclosure, the frequency of administration (e.g. of ingestion and/or digestion) can depend on several factors, including the desired level of effect. Generally, a regimen includes administration of the composition once or twice daily to include an administration in the morning and/or an administration in the evening. The amount of composition administered may depend on several factors, including level of desired results and the specific composition.

The follow examples, illustrating the compositions, methods, and uses of this disclosure, are intended to illustrate and not to limit the invention.

### EXAMPLES

Each probiotic strain is evaluated for its individual safety. Safety evaluations are just as important for multiple-strain probiotic products as for single strain probiotic products. For example, pathogenic Escherichia coli 0157:H7 apparently causes diarrhea while probiotic Escherichia coli Nissle 1917 reportedly reduces diarrhea (Korada et al. 2019). Since different strains may have such different effects, different combinations ofdifferent strains which are all potentially affecting gut transit times, abdominal symptoms such as bloating, pain, distension, rumbling, cramps, nausea or vomiting, could have different interacting effects. Not all clinical studies on probiotics systematically report risks associated with probiotic strains (Doron and Snydman 2015); more adverse event reporting is warranted. Therefore, the present combination was tested in a prospective tolerability study, NCT04044144. The study was a single arm, open-label study providing 105 billion CFU/day ofthe combination described in one capsule per day for 10 days to 10 healthy adults. The primary outcome was the frequency of new adverse events or serious adverse events. The secondary outcomes evaluated any new abnormal values on a complete blood count (CB3C) and comprehensive metabolic panel (CMIP). The exploratory outcomes evaluated changes in specific microbiota populations and short chain fatty acids.

Several examples of the composition are provided hereafter. The following formulations of the composition are provided based on a single capsule formula. In Tables 1 and 2 below, “CFU” refers to “colony forming units”, preferably as a “per capsule” dose as indicated. The target CFU per strain associated with clinical studies supporting the CFU per strain are listed in Table 1 and Table 2.

TABLE 1
Composition of the formula and additional possible combinations and amounts

| | | | | | Preferred | More | Most | Example 1 |
|---|---|---|---|---|---|---|---|---|
| | | | | Billion | Billion | Preferred | Preferred | Billion |
| | | | | CFU | CFU | Billion CFU | Billion CFU | CFU |
| Genus | Species | Strain | ATCC | per dose* | per dose | per dose* | per dose* | per capsule |
| Lactobacillus | acidophilus | NCFM | 5221 | 1 | 7 | 20 | 100 | 12.5 |
| Bifidobacterium | lactis | Bi-07 | 5220 | 4.25 | 5 | 14 | 20 | 12.5 |
| Bifidobacterium | lactis | Bl-04 | 5219 | 2 | 4.25 | 5 | 20 | 20 |
| Lactobacillus | plantarum | Lp-115 | 5209 | 1 | 5 | 10 | 20 | 20 |
| Lactobacillus | rhamnosus | GG | 7017 | 5 | 6 | 10 | 20 | 20 |
| Lactobacillus | rhamnosus | HN001 | 5675 | 1 | 5-6 | 25 | 50 | 5 |
| Bifidobacterium | lactis | HN019 | 5674 | 2 | 5 | 17-17.2 | 27.2-30 | 5 |
| Lactobacillus | paracasei | Lpc-37 | 5275 | 4.25 | 5 | 10 | 17 | 10 |

In view ofTable 1 above, it should be appreciated that the composition ofthis disclosure caninclude eachofthe individual billion CFU per dose amounts for each strain/ATCC of bacteria, e.g. as a minimum, a lower boundary, or an “at least” amount, or as a range established between specific amounts for a given probiotic or combination or probiotics. In addition, different combinations and ranges for each probiotic can be provided by selecting numbers from the same or different rows and/or columns. Suitable probiotics for the composition of this disclosure are commercially available from various sources, such as from ATCC®.

TABLE 2
Composition of the formula and additional possible combinations and amounts

| | | | | Billion | Preferred | More | Most | Billion |
|---|---|---|---|---|---|---|---|---|
| | | | | CFU | Billion | Preferred | Preferred | CFU |
| | | | | per | CFU | Billion CFU | Billion CFU | per |
| Genus | Species | Strain | ATCC | dose* | per dose | per dose* | per dose* | capsule |
| Lactobacillus | acidophilus | NCFM | 5221 | 1 | 7 | 20 | 100 | 12.5 |
| Bifidobacterium | lactis | Bi-07 | 5220 | 4.25 | 5 | 14 | 20 | 12.5 |
| Bifidobacterium | lactis | Bl-04 | 5219 | 2 | 4.25 | 5 | 20 | 20 |
| Lactobacillus | plantarum | 299v | 14917T | 1 | 5 | 10 | 20 | 20 |
| Lactobacillus | rhamnosus | GG | 7017 | 5 | 6 | 10 | 20 | 20 |
| Lactobacillus | rhamnosus | HN001 | 5675 | 1 | 5-6 | 25 | 50 | 5 |
| Bifidobacterium | lactis | HN019 | 5674 | 2 | 5 | 17-17.2 | 27.2-30 | 5 |
| Lactobacillus | paracasei | Lpc-37 | 5275 | 4.25 | 5 | 10 | 17 | 10 |

In view of Table 2 above, it should be appreciated that the composition of this disclosure can include each of the individual billion CFU per dose amounts for each strain/ATCC of bacteria, e.g. as a minimum, a lower boundary, or an “at least” amount, or as a range established between specific amounts for a given probiotic or combination or probiotics. In addition, different combinations and ranges for each probiotic can be provided by selecting numbers from the same or different rows and/or columns.

Composition of the disclosure having a synergistic combination of strains:

This product is a first of its kind product to combine these specific bacteria in a single formula and they may work in a synergistic way to cross-feed other bacteria through their metabolite production, thereby affecting other populations of bacteria in the intestine.

One might expect an increase in the stool in Lactobacillus species and Bifidobacteria species which are present in the composition. However, an increase in the clinically-relevant strains Akkermansia muciniphila and F. prausnitzii is entirely unexpected. This unexpected increase in these other intestinal bacteria are associated with health providing a novel approach for modulating microbiome diversity in a manner that circumvents the challenge of manufacturing sufficient quantities of Akkermansia muciniphila and F. prausnitzii species as commercially available probiotic dietary supplements.

The composition is a blend and then encapsulated. Hard-shell capsules filled with active ingredients and excipients, including but not limited to microcrystalline cellulose, and then sealed with hard shell caps including typical clear vegetable caps and hydroxypropyl methylcellulose or hypromellose (HPMC). Lubricants such as magnesium stearate, silica or silicon dioxide, or stearic acid can be used to improve rheological properties of product powders during manufacturing.

Such excipients are typically used in an amount of from 0.1 to 140 mg, or optionally in an amount of approximately 130 mg total.

Uses of the Composition of the Disclosure:

Prior art on these strains show clinical evidence for various gastrointestinal and immune benefits, which recommends this formula for individuals with means of improving gastrointestinal and immune and metabolic health through stress resilience such as addressing low bacterial diversity or dysbiosis conditions associated with inflammatory and metabolic dysregulation. In addition, it demonstrates the ability to improve Firmicutes/Bacteroidetes ratio, modify the intestinal microbiome's diversity indices, to increase populations of specific bacteria associated with health, and to produce beneficial metabolites and interact with immune cells in the intestine.

Thus the formula is proposed for irregularity, poor frequency of stools, resolving loose stools to more formed stools, irritable bowel syndrome, constipation, diarrhea, flatulence, bloating, modulating inflammation of the intestine associated with diseases such as inflammatory bowel disease including Crohn's or Ulcerative colitis and the manifestation of pouchitis, celiac disease and other intestinal disorders, malabsorption and digestive disorders, abdominal pain, cramping, abdominal motility issues, compromised immune function including increased susceptibility or occurrence of upper respiratory tract infections/the common cold, adjuvant support for immunizations or any immunotherapy, allergen challenges, environmental toxins, individuals particularly susceptible to stress-induced headaches and abdominal pain, and/or relief from anxiety or depression comorbid with gastrointestinal distress.

Testing of the Composition of Example 1:

In a small, open-label study of 10 healthy individuals consuming one capsule of the formulation each day for 10 days (NCT04044144), it was discovered that several bacteria associated with health were increased. Eight subjects' data were collected (see Table 3).

TABLE 3
Baseline and Endpoint (day 10*) measurements of bacteria levels by qPCR correlation to
CFU in stool of participants consuming the composition of this disclosure. Scientific notation used
in these tables, e.g., 6.50E+07 is 6.5 × 10\hat{ }7 or 65,000,000. < signifies that the quantity present
was below the detection limit, specified numerically for each test. In the tables below amounts are
expressed as colony forming units per gram of stool (CFU/g stool). The table below is split into
two parts in consideration of space.

| Faecalibacterium prausnitzii | | | | Lactobacillus spp. | | | |
|---|---|---|---|---|---|---|---|
| Baseline (=0 days) | | End (=10* days) | | Baseline | | End | |
| 9.1E+08 | | 2.3E+09 | | 2.2E+09 | | 3.8E+09 | |
| 3.0E+08 | | 6.9E+09 | | 1.4E+09 | | 2.0E+10 | |
| 3.0E+09 | | 2.1E+09 | | 1.4E+09 | | 5.3E+09 | |
| <1.2E+07 | | 2.5E+09 | | 6.1E+07 | | 5.9E+09 | |
| 1.2E+09 | | 2.1E+09 | | 8.9E+08 | | 3.7E+09 | |
| 2.8E+09 | | 1.2E+10 | | 1.5E+09 | | 2.0E+10 | |
| 1.2E+09 | | 1.4E+10 | | 5.8E+07 | | 3.8E+09 | |
| 7.5E+09 | | 1.8E+10 | | 3.8E+09 | | 1.4E+10 | |
| Ruminococcus | | Bifidobacterium | | Akkermansia | | Desulfovibrio | |
| spp. | | spp. | | muciniphila | | piger | |
| Baseline | End | Baseline | End | Baseline | End | Baseline | End |
| 1.2E+08 | 6.1E+07 | 1.3E+09 | 8.5E+08 | <5.6E+04 | 8.6E+05 | 3.9E+05 | <1.0E+04 |
| 6.5E+07 | 7.5E+08 | 1.6E+08 | 1.1E+09 | <5.6E+04 | 3.1E+07 | <1.0E+04 | <1.0E+04 |
| 1.6E+08 | 1.7E+08 | 1.9E+09 | 5.7E+09 | 2.0E+07 | 3.2E+07 | 9.4E+07 | 6.80E+07 |
| 6.4E+06 | 5.2E+07 | <3.9E+06 | 6.3E+08 | <5.6E+04 | 6.1E+05 | 3.2E+06 | 1.30E+05 |
| 9.0E+07 | 1.4E+08 | 1.8E+09 | 1.9E+09 | 1.4E+07 | 1.8E+07 | 2.9E+05 | <1.0E+04 |
| 2.2E+08 | 3.6E+08 | 7.2E+08 | 5.2E+08 | 6.1E+06 | 4.8E+06 | <1.0E+04 | <1.0E+04 |
| 1.8E+08 | 8.8E+08 | 6.9E+08 | 2.9E+09 | <5.6E+04 | 1.5E+07 | 2.8E+05 | <1.0E+04 |
| 4.3E+08 | 2.2E+09 | 5.9E+09 | 1.0E+10 | 4.9E+07 | 5.5E+07 | <1.0E+04 | <1.0E+04 |

*For exact number of days per subject, listed in chronological order from top to bottom of chart, refer to Table 5.

Measurements by qPCR using Genova Diagnostics GI Effects profile test. (Chen et al. 2019). Lactobacillus and Bifidobacteria were present in the probiotic composition, and appeared in the stool analysis. However, levels of Faecalibacterium prausnitzii, Akkermansia muciniphila, and Ruminococcaceae species, which were not present in the probiotic composition, also increased. Firmicutes/Bacteroidetes ratio changed over the course of the study (see Table 3). At baseline, half of the study participants had a Firmicutes/Bacteroidetes ratio that was outside of the laboratory reference range (low). At study end, all participants had a Firmicutes/Bacteroidetes ratio that was within the laboratory reference (normal). Thus, by this measure using the reference range of the test lab, and others, a measurable indicator of dysbiosis is improved by the composition of this disclosure.

TABLE 4
Baseline and Endpoint (day 10*) measurements of Firmicutes/Bacteroidetes
ratio in stool of participants consuming the composition of this disclosure
Firmicutes/Bacteroidetes Ratio

| Baseline (=0 days) | | End (=10* days) | |
|---|---|---|---|
| Ratio | Within Reference Range** | Ratio | Within Reference Range |
| 15 | Normal | 18 | Normal |
| 5 | Low | 20 | Normal |
| 9 | Normal | 13 | Normal |
| 1 | Low | 12 | Normal |
| 16 | Normal | 16 | Normal |
| 8 | Low | 18 | Normal |
| 3 | Low | 16 | Normal |
| 25 | Normal | 27 | Normal |

*Refer to Table 5 for the number of days the product was consumed.
**Reference Range as indicated by Genova Diagnostics.

The improvement in the ratio was potentially related to the increases in bacteria within the Firmicutes phylum: Lactobacillus species, Faecalibacterium prausnitzii, and Ruminococcaceae species.

Akkermansia muciniphila was not present at detectable levels in some individuals at the initiation of the study, but after consumption of the composition of this disclosure, it was detectable, and in those individuals who had detectable levels at the outset, it was found in much higher abundance. Thus, by this measure, and others, a measurable diversity index is improved by the composition ofthis disclosure.

Clinical Observations of Improved Symptomology

The 10 subjects completed a questionnaire on symptoms at baseline and after 10*days of consuming one capsule each day of the formula in example 1. The questionnaire results and additional comments provided by the subjects showed the changes in symptoms as described above.

TABLE 5

| | # | Symptom(s) Changed | Relative Change |
|---|---|---|---|
| Subject | days* | (Questionnaire Quantification) | (Baseline to End) |
| 1 | 9 | Increased Frequency of Bowel | ++ |
| | | Movements (BMs) | |
| 2 | 10 | Nausea | ++++ |
| 3 | 10 | Decreased Appetite | ++ |
| 4 | 10 | Increased frequency of bowel movement | + |
| | | Loose stools (reduced) | + |
| | | Urgent need to pass BM (reduced) | + |
| | | Increased level of energy | + |
| 5 | 12 | Increase in energy | +++ |
| | | Increased frequency of bowel movements | + |
| | | Diarrhea | + |
| 6 | 10 | No change in Questionnaire results | 0 |
| 7 | 10 | Rumbling in the abdomen (improved) | ++ |
| | | Abdominal pain (decreased) | + |
| | | Bloating of the abdomen (reduced) | + |
| | | Flatulence or gas (reduced) | ++ |
| | | Sensation of not completely emptying | + |
| | | bowels (reduced) | |
| | | Loose stools | ++ |
| | | Difficulty falling asleep | ++ |
| | | Increased energy | ++ |
| | | Decreased appetite | ++ |
| | | Seasonal allergies (reduced) | + |
| | | Runny nose | + |
| | | Muscle aches or pain (reduced) | +++ |
| 8 | 10 | Flatulence or gas (reduced) | + |
| | | Loose stools | + |
| | | Urgent need to pass a bowel | +++ |
| | | movement (reduced) | |
| | | Difficulty falling asleep (reduced) | +++ |
| | | Muscle aches or pain (reduced) | ++++ |
| 9 | 9 | Flatulence or gas (reduced) | ++ |
| | | Hard stools (reduced) | ++ |
| | | Loose stools (reduced) | +++ |
| 10 | 10 | Heartbum (reduced) | + |
| | | Rumbling in the abdomen | ++ |
| | | Abdominal pain or cramps (reduced) | ++ |
| | | Flatulence or gas | + |
| | | Mouth sores | ++ |
| | | Difficulty falling asleep | ++ |
| | | Headaches | +++ |
| | | Sore throat | + |

*There were slight variations in number of capsules taken for the whole study

Example 2-In vivo study

Evaluation of this probiotic composition's impact on the gut microbiome and metabolome in animals performed as described in a previous study of measuring microbiome and metabolome impact (Li et al. 2019) with expanded diversity analyses described previously (Finotello, Mastrorilli, and Di Camillo 2016). Findings showed that changes in bacteria in the gut of the animal model corresponded with beneficial metabolite production, thus demonstrating a potential therapeutic for modulating the intestinal environment and physiological outcomes.

Further evaluation of the probiotic composition in the context of malnourished and diarrhea-stricken animals demonstrated improved absorption of nutrients, decreased inflammation, and improved intestinal permeability as demonstrated previously (de Queiroz et al. 2014). Findings suggest therapeutic applications for diarrhea from antibiotic usage, travel, intestinal hyper-permeability/leaky gut, inflammatory bowel disease or inflammation in the gut and malnourishment due to impaired intestinal function.

Example 3—In vitro study

Probiotic composition evaluated within the context of feces derived from healthy or diseased/dysbiotic individuals demonstrated changes in microbial populations through incubation.

Conducted as described previously (Forssten and Ouwehand 2017), measurements showed that potentially pathogenic bacteria decreased while beneficial bacteria increased or stayed the same.

In addition, production of beneficial metabolites such as nutrients, neurotransmitters such as serotonin or short chain fatty acids increased in the incubation culture as described in this model (Li et al. 2019).

The individual strains and/or the formula combination have been demonstrated to disrupt a layer of biofilm, particularly of pathogenic bacteria, present on a layer of intestinal epithelial cells as previously described (Kaur et al. 2018).

Example 4—Clinical Use of the Composition in Microbiotic Repopulation

A 47 year old male presented after foreign travel with continuing “traveler's diarrhea.” After a course of antibiotics, and screening for parasites, the patient continued to exhibit symptoms. The patient takes a daily dose of Example 1 of the composition. Symptoms subside within a week.

Based upon these results, it is expected related stresses on the microbiome, such as surgical procedure(s), stress-induced by travel, antibiotics, or other lifestyle factors, environmental or psychological displaying symptoms such as tendency to respiratory tract infections, allergies, skin or atopic disease, pain after colonoscopy or surgical procedures may be treated by consuming a course of the probiotic composition of this disclosure. Immune response due to the stress of an allergen (L.-Z. Xu et al. 2016), atopic disease and other skin-related irritations (Larsen et al. 2011) or food intolerances (Canani et al. 2012), amelioration of abdominal pain via questionnaires, additionally for IBS (Ducrotté, Sawant, and Jayanthi 2012), reduction in severity and duration of antibiotic-associated and other stress-induced diarrhea (Dudzicz et al. 2018) and decreased cold and flu-like symptoms (S. Liu et al. 2013) demonstrated the stress resilience by consuming the composition of this disclosure.

Example 5—Clinical study

In a group of heterogeneous adults including those with intestinal hyperpermeability or GI distress with dysbiosis or low diversity, treated in an n-of-1 design (Kravitz and Duan 2014, 1) to evaluate effects with and without the probiotic composition, 1 dose per day containing the formulation for a period of 1 week, 10 days, 2 weeks, 1 month, 3 months and/or 6 months, measurements were performed pre and post intervention, with alternating periods of intervention and control, as follows:

Stool analysis using either 16S ribosomal rRNA sequencing or shotgun sequencing to identify bacteria, characterizing the relative abundance, diversity using indices (Finotello, Mastrorilli, and Di Camillo 2016), Measurements of metabolites and SCFA including but not limited to acetate, propionate, butyrate, isovalerate, using metabolome techniques previously described (Li et al. 2019), Wellbeing questionnaires and validated questionnaire on satiety, anxiety, stress, constipation, diarrhea, gas and bloating (PROMIS questionnaires). Findings showed amelioration of symptoms in several use cases depending on patient complaints at baseline, including improvement in IBS, reduction in intestinal hyperpermeability scores via lactulose mannitol testing (Musa et al. 2019) and other testing. Additional group comparisons demonstrated similar GI and immune benefits.

Example 6—Clinical Study on Stress and Allergen/Environmental Challenges

In a group of sensitive individuals prone to reacting to environmental challenges such as seasonal allergies and stress-induced fatigue, 1 dose per day containing the formulation for a period of 1 week, 10 days, 2 weeks, 1 month, 3 months and/or 6 months, measurements were performed pre and post intervention, with alternating periods of intervention and control, as follows:

Stool analysis using either 16S ribosomal rRNA sequencing or shotgun sequencing to identify bacteria, characterizing the relative abundance (Maier et al. 2017), diversity using indices (Finotello, Mastrorilli, and Di Camillo 2016), and measurements of SCFA including but not limited to acetate, propionate, butyrate, isovalerate, using metabolome techniques previously described (Li et al. 2019; Maier et al. 2017) demonstrated changes in diversity, amelioration of dysbiosis, and beneficial changes in the intestinal environment. Wellbeing questionnaires and validated questionnaire on satiety, anxiety, stress, constipation, diarrhea, gas and bloating (PROMIS questionnaires) demonstrated resilience to environmental and life stressors. Blood biomarkers including immune cells, cytokines, immunoglobulins (Paineau et al. 2008) including but not limited to IgG subclasses(Zhang et al. 2015), liver enzymes, and phagocytic or natural killer cell activity assays (H. S. Gill et al. 2000) demonstrating immune responses or detoxification pathways through the liver showed stress resilience effects. Findings showed amelioration of symptoms in several use cases depending on patient complaints at baseline, including reduction in symptoms compared to placebo or non-use of the material, and improvement in immune response via immunological markers from serum of the individuals (Harsharnjit S. Gill et al. 2001) or by in vitro assessment of fecal samples from the individuals (Y. Liu, Gibson, and Walton 2016).

The skilled artisan will appreciate the utility of the composition described above, and reasonable variations thereof. The composition is useful in treating dysbiosis manifesting itself in a number of ways from gastric distress to IBS. As demonstrated, it produces clinical results such as relief from bloating, increased stool regularity, reduced appetite, more formed stool, decreased abdominal pain, mitigated perceived feelings of stress, and/or low mood such as anxiety or depression associated with gastrointestinal distress.

### Additional Embodiments

The following additional embodiments are provided, the numbering of which is not to be construed as designating levels of importance. Moreover, it is to be understood that the embodiments recited below are provided in conjunction with and in addition to the embodiments described above, as well as those claimed further below. Thus, it is also to be understood that variations, combinations, and/or modifications of the embodiment(s) and/or feature(s) of the embodiment(s) may be within the scope of the present invention. Likewise, alternative embodiments that result from combining, integrating, and/or omitting features of the embodiment(s) described herein may also be within the scope of the present invention.

Embodiment 1 relates to a probiotic composition comprising: Bifidobacterium lactis, Lactobacillus acidophilus, Lactobacillus plantarum, Lactobacillus rhamnosus, Lactobacillus rhamnosus, Bifidobacterium lactis, and Lactobacillus paracasei.

Embodiment 2 relates to the probiotic composition according to Embodiment 1, comprising: Bifidobacterium lactis ATCC 5220, Lactobacillus acidophilus ATCC 5221, Bifidobacterium lactis ATCC 5219, Lactobacillus plantarum ATCC 5209 or ATCC 14917T, Lactobacillus rhamnosus ATCC 7017, Lactobacillus rhamnosus ATCC 5675, Bifidobacterium lactis ATCC 5674, and Lactobacillus paracasei ATCC 5275.

Embodiment 3 relates to the probiotic composition according to Embodiment 1, comprising: Bifidobacterium lactis Bi-07, Lactobacillus acidophilus NCFM, Bifidobacterium lactis B1-04, Lactobacillus plantarum Lp-115 or 299v, Lactobacillus rhamnosus GG, Lactobacillus rhamnosus HN001, Bifidobacterium lactis HN019, and Lactobacillus paracasei Lpc-37.

Embodiment 4 relates to the probiotic composition according to Embodiment 1 having the ratios of probiotics as follows: Bifidobacterium lactis, Lactobacillus rhamnosus, Lactobacillus plantarum, Lactobacillus acidophilus, and Lactobacillus paracasei (75: 50: 40: 25: 20).

Embodiment 5 relates to the probiotic composition according to Embodiment 1, having the CFU at expiration date of each of the probiotics as follows; 12.5 billion CFU Bifidobacterium lactis ATCC 5220, 12.5 billion CFU Lactobacillus acidophilus ATCC 5221, 20 billion CFU Bifidobacterium lactis ATCC 5219, 20 billion CFU Lactobacillus plantarum ATCC 5209 or ATCC 14917T, 20 billion CFU Lactobacillus rhamnosus ATCC 7017, 5 billion CFU Lactobacillus rhamnosus ATCC 5675, 5 billion CFU Bifidobacterium lactis ATCC 5674, and 10 billion CFU Lactobacillus paracasei ATCC 5275.

Embodiment 6 relates to the use of the probiotic composition of any one of Embodiments 1-5, in the treatment of a disorder selected from the group consisting of: irregularity, poor frequency of stools, resolve loose stools to more formed stools, irritable bowel syndrome, constipation, diarrhea, flatulence, bloating, inflammation of the intestine such as inflammatory bowel disease including Crohn's or Ulcerative colitis and the manifestation of pouchitis, celiac disease and other intestinal disorders, malabsorption and digestive disorders, abdominal pain, cramping, abdominal motility issues, compromised immune such as increased susceptibility or occurrence of upper respiratory tract infections/the common cold, adjuvant support for immunizations, allergen challenges, environmental toxins, individuals particularly susceptible to stress-induced headaches and abdominal pain, and/or relief from anxiety or depression comorbid with gastrointestinal distress.

Embodiment 7 relates to the use of the probiotic composition of any one of Embodiments 1-5, to create enhanced microbiome diversity.

Embodiment 8 relates to the use of the probiotic composition of any one of Embodiments 1-5, to increase species Akkermansia muciniphila and Faecalibacterium prausnitzii and Ruminococcus spp.

Embodiment 9 relates to the use of the probiotic composition of any one of Embodiments 1-5, to improve Firmicutes/Bacteroidetes ratio and decrease dysbiosis.

Embodiment 10 relates to the use of the probiotic composition of any one of Embodiments 1-5, to decrease dysbiosis and increase diversity in the intestine, inhibiting the growth of potentially pathogenic bacteria and increasing levels of beneficial bacteria.

Embodiment 11 relates to the use of the probiotic composition of any one of Embodiments 1-5, to increase levels of beneficial metabolites of bacteria including short chain fatty acids.

Embodiment 12 relates to the use of the probiotic composition of any one of Embodiments 1-5, to resolve antibiotic-associated diarrhea or traveler's diarrhea.

Embodiment 13 relates to the use of the probiotic composition of any one of Embodiments 1-5, to treat inflammatory bowel disease.

Embodiment 14 relates to the use of the probiotic composition of any one of Embodiments 1-5, to decrease appetite.

Embodiment 15 relates to the use of the probiotic composition of any one of Embodiments 1-5, to decrease symptoms associated with allergic response or irritation by environmental allergens and pollutants.

Embodiment 16 relates to the use of the probiotic composition of any one of Embodiments 1-5, to strengthen the function of the gut barrier and improve intestinal permeability.

Embodiment 17 relates to the use of the probiotic composition of any one of Embodiments 1-5, to improve the response to stress.

Embodiment 18 relates to the use of the probiotic composition of any one of Embodiments 1-5, to increase absorption of nutrients.

Embodiment 19 relates to the use of the probiotic composition of any one of Embodiments 1-5, to increase production of nutrients by the bacteria in the intestine.

Embodiment 20 relates to the use of the probiotic composition of any one of Embodiments 1-5, to promote neurotransmitter production.

Embodiment 21 relates to the use of the probiotic composition of any one of Embodiments 1-5, as a medical food to manage the nutritional needs to support the microbial diversity for irregularity, poor frequency of stools, resolve loose stools to more formed stools, irritable bowel syndrome, constipation, diarrhea, flatulence, bloating, inflammation of the intestine such as inflammatory bowel disease including Crohn's or Ulcerative colitis and the manifestation of pouchitis, celiac disease and other intestinal disorders, malabsorption and digestive disorders, abdominal pain, cramping, abdominal motility issues, compromised immune such as increased susceptibility or occurrence of upper respiratory tract infections/the common cold, adjuvant support for immunizations, allergen challenges, environmental toxins, individuals particularly susceptible to stress-induced headaches and abdominal pain, and/or relief from anxiety or depression comorbid with gastrointestinal distress.

Each of the additional embodiments so defined may be combined with any other embodiment or aspect of the embodiments of the invention described herein. In particular, any feature indicated as being optional or advantageous may be combined with any other feature or features indicated as being optional or advantageous, and each aspect of embodiments of the composition are to be understood as being applicable to use in the embodiments of the methods of using the composition.

The terms “comprising” or “comprise” are used herein in their broadest sense to mean and encompass the notions of “including,” “include,” “consist(ing) essentially of,” and “consist(ing) of.” The use of “for example,” “e.g.,” “such as,” and “including” to list illustrative examples does not limit to only the listed examples. Thus, “for example” or “such as” means “for example, but not limited to” or “such as, but not limited to” and encompasses other similar or equivalent examples. The term “about” as used herein serves to reasonably encompass or describe minor variations in numerical values measured by instrumental analysis or as a result of sample handling. Such minor variations may be in the order of 0-25, +0-10, +0-5, or ±0-2.5, % of the numerical values. Further, The term “about” applies to both numerical values when associated with a range of values. Moreover, the term “about” may apply to numerical values even when not explicitly stated.

Generally, as used herein a hyphen “-” or dash “-” in a range of values is “to” or “through”; a “>” is “above” or “greater-than”; a “>” is “at least” or “greater-than or equal to”; a “<” is “below” or “less-than”; and a “<” is “at most” or “less-than or equal to.” On an individual basis, each of the aforementioned applications for patent, patents, and/or patent application publications, is expressly incorporated herein by reference in its entirety in one or more non-limiting embodiments.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, it is to be appreciated that different, special, and/or unexpected results may be obtained from each member of the respective Markush group independent from all other Markush members. Each member of a Markush group may be relied upon individually and or in combination and provides adequate support for specific embodiments within the scope of the appended claims.

It is also to be understood that any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

1. A probiotic composition comprising: Bifidobacterium lactis; Lactobacillus acidophilus; Lactobacillus plantarum; Lactobacillus rhamnosus; and Lactobacillus paracasei.

2. The probiotic composition according to claim 1, comprising at least two different strains of Lactobacillus rhamnosus.

3. The probiotic composition according to claim 1,
comprising at least two different strains of Bifidobacterium lactis.

4. The probiotic composition according to claim 3, comprising at least three different strains of Bifidobacterium lactis.

5. The probiotic composition according to claim 1, comprising: Bifidobacterium lactis ATCC 5220; Lactobacillus acidophilus ATCC 5221;
Bifidobacterium lactis ATCC 5219; at least one Lactobacillus plantarum selected from the group consisting of Lactobacillus plantarum ATCC 5209 and Lactobacillus plantarum ATCC 14917T;
Lactobacillus rhamnosus ATCC 7017; Lactobacillus rhamnosus ATCC 5675; Bifidobacterium lactis ATCC 5674; and Lactobacillus paracasei ATCC 5275.

6. The probiotic composition according to claim 5, comprising Lactobacillus plantarum ATCC 5209.

7. The probiotic composition according to claim 5, comprising Lactobacillus plantarum ATCC 14917T.

8. The probiotic composition according to claim 1, comprising: Bifidobacterium lactis Bi-07; Lactobacillus acidophilus NCFM; Bifidobacterium lactis B1-04; at least one Lactobacillus plantarum selected from the group consisting of Lactobacillus plantarum Lp-115 and Lactobacillus plantarum 299v; Lactobacillus rhamnosus GG; Lactobacillus rhamnosus HN001; Bifidobacterium lactis HN019; and Lactobacillus paracasei Lpc-37.

9. The probiotic composition according to claim 8, comprising Lactobacillus plantarum Lp-115.

10. The probiotic composition according to claim 8, comprising Lactobacillus plantarum 299v.

11. The probiotic composition according to claim 1, having a CFU ratio of each of the probiotics as follows: Bifidobacterium lactis, Lactobacillus rhamnosus, Lactobacillus plantarum, Lactobacillus acidophilus, and Lactobacillus paracasei (75: 50: 40: 25: 20).

12. The probiotic composition according to claim 1, having the CFU at expiration date of each of the probiotics as follows: at least 12.5 billion CFU Bifidobacterium lactis ATCC 5220; at least 12.5 billion CFU Lactobacillus acidophilus ATCC 5221; at least 20 billion CFU Bifidobacterium lactis ATCC 5219; at least 20 billion CFU Lactobacillus plantarum ATCC 5209 or at least 20 billion CFU Lactobacillus plantarum ATCC 14917T; at least 20 billion CFU Lactobacillus rhamnosus ATCC 7017; at least 5 billion CFU Lactobacillus rhamnosus ATCC 5675; at least 5 billion CFU Bifidobacterium lactis ATCC 5674; and at least 10 billion CFU Lactobacillus paracasei ATCC 5275.

13. A foodstuff or beverage for use in treating and/or preventing dysbiosis and/or maladies related thereto, wherein the foodstuff or beverage comprises the probiotic composition according to claim 1.

14. The foodstuff or beverage of claim 13, further defined as a medical food.

15. A method of treating a disorder, the method comprising administering the composition of claim 1 to a subject, wherein the disorder is selected from the group consisting of: irregularity, poor frequency of stools, resolve loose stools to more formed stools, irritable bowel syndrome, constipation, diarrhea, flatulence, bloating, inflammation of the intestine such as inflammatory bowel disease including Crohn's or Ulcerative colitis and the manifestation of pouchitis, celiac disease and other intestinal disorders, malabsorption and digestive disorders, abdominal pain, cramping, abdominal motility issues, compromised immune such as increased susceptibility or occurrence of upper respiratory tract infections/the common cold, adjuvant support for immunizations, allergen challenges, environmental toxins, individuals particularly susceptible to stress-induced headaches and abdominal pain, relief from anxiety or depression comorbid with gastrointestinal distress, and combinations thereof.

16. A method comprising administering the composition of claim 1 to a subject to at least one of:
i) create enhanced microbiome diversity; and/or
ii) increase species Akkermansia muciniphila and Faecalibacterium prausnitzii and Ruminococcus spp.; and/or
iii) improve Firmicutes/Bacteroidetes ratio and decrease dysbiosis; and/or
iv) decrease dysbiosis and increase diversity in the intestine, inhibiting the growth of potentially pathogenic bacteria and increasing levels of beneficial bacteria; and/or
v) increase levels of beneficial metabolites of bacteria including short chain fatty acids; and/or
vi) resolve antibiotic-associated diarrhea or traveler's diarrhea; and/or
vii) treat inflammatory bowel disease; and/or
viii) decrease appetite; and/or
ix) decrease symptoms associated with allergic response or irritation by environmental allergens and pollutants; and/or
x) strengthen the function of the gut barrier and improve intestinal permeability; and/or
xi) improve the response to stress; and/or
xii) increase absorption of nutrients; and/or
xiii) increase production of nutrients by the bacteria in the intestine; and/or
xiv) promote neurotransmitter production.

17. (canceled)
